(19) United States
(12) Patent Application Publication
(10) Pub. No.: US 2021/0282894 A1
(43) Pub. Date: Sep. 16, 2021

(54) DRILL JIG FOR AN ORAL SURGERY INTERVENTION

(71) Applicant: DANUBE PRIVATE UNIVERSITY GMBH, Krems-Stein (AT)

(72) Inventor: Constantin Von See, Imbach/Krems (AT)

(21) Appl. No.: 17/262,557

(22) PCT Filed: Jul. 24, 2019
(86) PCT No.: PCT/EP2019/069914

§ 371 Date: Jan. 22, 2021

(30) Foreign Application Priority Data
Jul. 24, 2018 (EP) 18185132.0

(51) Int. Cl. A61C 1/08; A61B 17/17; A61C 13/107; A61C 8/00

(52) U.S. Cl. CPC A61C 1/084; A61B 17/176; A61C 8/008; A61C 8/0089; A61C 13/0001

(57) ABSTRACT

The invention relates to a drill jig (1) for an oral surgery intervention in a patient, said drill jig (1) having a temporary dental device (5) in which a drill guide (3) for guiding a drill in the jaw of the patient is provided, and at least one holding element (4), connected to the temporary dental device (5), for temporarily fastening the drill jig (1) to the set of teeth (2) of the patient.

The invention relates to a drill jig for an oral surgery intervention on a patient.

Drill jigs are used in oral surgery interventions for guiding drills or other dental instruments. For this purpose, drill jigs usually have drill guides, through which the drill is guided to the position to be treated and a drill hole set at an angle predetermined by the drill jig can be created. Generally, in this case, not only does the drill jig prevent an inadvertent displacement of the drill during the intervention, rather it also specifies the precise position of the drill hole in the jaw of a patient.

Drill jigs are important during the fitting of dental implants in particular, as their correct position is decisive for the aesthetic and mechanical rehabilitation of the patient. As an implant can be fitted in the jaw in six degrees of freedom, the correct positioning of the dental implant or of the drill hole represents a great challenge. In the region of the front teeth in particular, drill jigs ensure better aesthetic results compared to implant fittings without a drill jig. By contrast, incorrectly or poorly positioned implants may make necessitate a considerable technical outlay and be connected with additional costs and, above all, suffering of the patient. They may also make oral hygiene at the dental implant more difficult and lead to inflammation reactions at the implant, which ultimately lead to a removal of the implant as a consequence. Therefore, even the planning and creation of drill jigs represent important parts of implant fitting. Therefore, implant fitting at least comprises the planning, the actual surgical intervention (with drilling and insertion of the implants) and the subsequent integration phase.

In order to be able to place the drill holes in the jaw required for the insertion of the dental implants correctly, so that the drill holes later offer sufficient hold due to optimum utilization of the available space and in particular do not injure any nerves, drill jigs are usually designed in a computer-assisted manner after preceding measurement of the jaw or the dentition of the patient and subsequently produced by removal or build-up methods.

DE 20 2012 005510 U1 relates to a reference jig supported on the mucous membrane or on bone for preparing and carrying out implantations, characterized in that the jig is produced from an X-ray visible material, and in that defined external contours and/or catches or projections are attached to the jig on the surfaces facing away from the mucous membrane, and in that the X-ray visibility of the jig is set up in such a manner that both the three-dimensional external contours and/or the X-ray visible internal contours of the jig and the bone structures can be seen simultaneously in the computed tomography, and in that the X-ray jig and the drill jig are identical.

US 2016/0074141 A1 relates to a dental prosthesis for permanently servicing a gap between teeth. It is disclosed in the document that the dental prosthesis has a drill channel, through which a drill can be guided. The dental prosthesis can be fastened to the neighbouring teeth during the intervention, by means of removable wings. After drilling, a screw can be inserted into the drill hole in the jaw of the patient, which screw holds the dental prosthesis. It is provided that the dental prosthesis remains long-term in the dentition of the patient beyond the integration phase of the screw (cf. e.g. paragraphs [0039] and [0040] of the document).

During the integration phase of the implants, the freshly fitted dental implant is usually protected by a temporary tooth, however, which can likewise be shaped and created in a computer-assisted manner in a separate production process. Generally, in addition to protecting the integrating implants and preventing tooth migration of adjacent teeth, the temporary tooth also additionally fulfils aesthetic purposes and allows at least cautious chewing during the integration phase.

US 2013/0316297 A1 discloses a drill jig with artificial teeth and drill channels, wherein mounts are provided for fixing to neighbouring teeth. The document does not teach that the artificial teeth can be separated from the mounts for fixing to the neighbouring teeth. A use of these artificial teeth as temporary teeth is neither disclosed nor obvious.

EP 2 425 796 A1 discloses an implantation aid assembly, having a drill jig, a marker plate, a jaw adapter and prosthetic teeth. The jaw adapter is adapted to the jaw of a patient with the aid of a rapidly curing plastic. A coupling plate with coupling pins, to which the marker plate is in turn fastened, is adhesively bonded onto the individual moulding. The marker plate contains a plurality of 3D markers made from metal, which should enable the three-dimensional detection of the entire arrangement on the jaw of the patient. After the detection of the X-ray implantation aid assembly, guide drill holes are planned and introduced into the drill jig. The marker plate is removed and the drill jig is directly connected to the jaw adapter. Subsequently, the jaw adapter is drilled through under guidance of the drill jig, so that guide drill holes are finally created. The drill jig is removed again and the jaw adapter, which is drilled through, then forms the drill jig for the implant fitting. The prosthetic teeth mentioned are used merely as place holders during the radiological detection. A use of these place holders as temporary teeth is neither disclosed nor obvious.

The known production processes for creating the drill jig and the temporary tooth are very time-consuming and connected with high costs. And although there is a high outlay in the prior art for planning and producing the drill jig and the temporary tooth, the temporary tooth sometimes does not exactly fit the dental implant fitted. It would therefore be desirable if the temporary tooth could be adapted more precisely to the dental implant fitted and the production outlay could be minimized. Likewise, it would be advantageous if the duration or complexity of the dental intervention for fitting the implants and the temporary tooth could be reduced.

The invention is therefore based on the object of providing a drill jig of the type mentioned at the beginning, which reduces or completely overcomes at least individual disadvantages of the prior art. The invention aims in particular to increase the fit between the temporary tooth and the dental implant and at the same time to reduce the production outlay of the temporary tooth, and/or to reduce the total duration or complexity of the dental treatment.

The stated object is achieved in this case in that the drill jig comprises a temporary tooth, in which a drill guide for guiding a drill into the jaw of the patient is provided, and at least one holding element, which is connected to the temporary tooth, for temporary fastening of the drill jig on the dentition of the patient; in particular, the temporary tooth is provided for temporary servicing of the patient during the wound healing phase (or integration phase) after the oral surgery intervention. Due to the combination according to the invention of the drill jig with the temporary tooth, both elements can be planned and created in a common production process, as a result of which the production outlay is reduced. In addition, the combination of the two elements previously separated from one another, inserted for different purposes and used in various phases of implant fitting, enables the improved adaptation of the temporary tooth to the drill hole and thus to the dental implant. Furthermore, the duration or complexity of the dental intervention is reduced owing to the combined fitting of the implants and the temporary tooth enabled by the invention.

In order to guide the drill into the jaw of the patient during the intervention, according to the invention, the temporary tooth comprises a drill guide which is preferably realized as a channel, which leads from a first side of the temporary tooth to a second side and penetrates the temporary tooth. Expediently, the channel is straight, i.e. free of curvature, and closed except for two end-side openings on the sides of the temporary tooth. The drill guide can be closed after drilling using a suitable material, in order to increase the aesthetics of the temporary tooth used. According to the invention, the temporary tooth is connected to at least one holding element, so that the temporary tooth or the drill jig can be fixed during the oral surgery intervention. The holding element prevents the inadvertent displacement of the temporary tooth or of the drill jig and can predetermine the orientation and positioning of the drill jig. Preferably, the holding element is adapted to the dentition of the patient for this purpose. If necessary, the holding element can also comprise a drill guide.

The drill jig preferably consists of a plastic or ceramic and can be manufactured in particular in one piece from a single material. It may however also be provided that the drill jig comprises various materials. For example, the temporary tooth may consist of a different material than the at least one holding element.

In principle, in the case of temporary teeth, it is possible to make a distinction between implant-supported and non-implant-supported temporary teeth. Implant-supported temporary teeth are already connected to the dental implant during the integration phase of the same. In order to protect the inserted dental implant, care should therefore be taken during the planning of implant-supported implants, that chewing contact with adjacent teeth is prevented and a material is used for the temporary tooth, which ensures a smallest possible force transmission into the dental implant. By contrast, non-implant-supported temporary teeth are not connected to the dental implant during the integration phase. Adhesive bridges (“Maryland bridges”) or provisional bridges to adjacent teeth are used in the prior art in order to nonetheless be able to fix the temporary tooth. In addition, there are non-implant-supported temporary teeth, which are realized as clasp prostheses and are fastened by means of a clasp to the roof of the mouth (palate) or to the adjacent teeth. The temporary tooth, which comprises the drill jig according to the invention, is preferably insertable or provided as an implant-supported or non-implant-supported temporary tooth.

For aesthetic and practical reasons, it is advantageous if the at least one holding element is connected to the temporary tooth by means of a detachable connection. Consequently, the holding element can easily be detached from the temporary tooth during the treatment of the patient by the dentist by hand or with the aid of a corresponding tool. Surplus parts can also be ground off using a file, if required. The temporary tooth can subsequently be placed at a position provided therefor, particularly adjacent to the dental implant inserted into the jaw of the patient.

For easier detachability, it is provided in a preferred embodiment, that the detachable connection is formed by a material constriction (tapering of material) or a perforation. As a result, the holding element can be detached from the temporary tooth particularly easily, for example by means of repeated bending.

In order to reduce the risk of an inadvertent displacement of the drill during the intervention further, it is beneficial if the at least one holding element is shaped for placement onto one or more teeth of the dentition of the patient for positioning the drill jig on the dentition of the patient. The hold of the holding element and therefore of the drill jig is increased due to the placement of the holding element onto one or more teeth. For further reduction of the production outlay, it is advantageous, particularly in this embodiment (but also in the whole context of the invention), if the holding element is constructed in one piece with the temporary tooth (and the same are preferably manufactured from a plastic or ceramic and/or preferably from a single material), for example by means of additive manufacturing.

In a further preferred embodiment, the at least one holding element comprises an opening, particularly an opening adapted to one or more teeth of the dentition of the patient, for positioning the drill jig on the dentition of the patient. This opening can be formed inter alia by means of an open cavity or a bulge of the holding element, which cavity or bulge on the inside, i.e. the side facing the dentition in the use state, represents a negative of one or more teeth of the patient. Due to this measure, the drill jig is particularly well secured against twisting or displacements during the intervention and an exact desired positioning is typically predetermined. For further reduction of the production outlay, it is advantageous, particularly in this embodiment (but also in the whole context of the invention), if the holding element is constructed in one piece with the temporary tooth (and the same are preferably manufactured from a plastic or ceramic and/or preferably from a single material), for example by means of additive manufacturing.

In order to not have to connect the temporary tooth to the freshly fitted dental implant during the integration phase, it is beneficial if the temporary tooth comprises at least one holding web for connection to a tooth surface, wherein the holding web is preferably formed by a connection section of the temporary tooth to the at least one holding element. Thus, the holding web may virtually function as a dental bridge to other teeth, wherein the bridge is also created during the production of the drill jig, so to say as part of the drill jig. Due to the holding web, the temporary tooth can be used particularly easily as a non-implant-supported temporary tooth. The holding web is expediently suited for connection to natural teeth adjacent to the dental implant and is preferably formed by connection elements protruding from the temporary tooth, such as wings, clips or caps for example. In addition, the holding web preferably consists of essentially solid, i.e. inflexible material. In particular, the entire drill jig can consist of the same material. During the dental treatment for placing the temporary tooth, the holding web can be provided with a suitable adhesive or plastic cement (or fastening plastic) and connected to the tooth surface of adjacent teeth. If required, the tooth surface of the adjacent teeth, particularly the enamel surface thereof, can be acid-etched beforehand. For easier production, the holding web can be formed by a connection section between the temporary tooth and the holding element, which connection section has a detachable connection at an end facing the holding element in particular.

So that implant-supported temporary teeth, i.e. temporary teeth which are already connected to the inserted implant during the integration phase, can be fastened to the implant, it is beneficial if the temporary tooth comprises a connection point for the indirect or direct connection to an implant on a side facing the jaw of the patient in the inserted state. Advantageously, the connection point can be adapted to the implant and comprise a thread, using which the temporary tooth can be connected to the implant. Preferably, in the state connected to the implant, the connection point enables an at least partial rotation of the temporary tooth relatively to the implant, in order to enable subsequent adjustments of the temporary tooth in relation to the position relative to the dentition of the patient. In the connected state, an intermediate piece can also additionally be arranged as an adapter between the implant and the connection point, wherein the connection point is adapted to the intermediate piece in this case. It is of course also conceivable to provide the temporary tooth simultaneously as implant-supported and non-implant-supported, i.e. both with a connection point, and with holding webs, as a result of which the dentist can choose, for example depending on the oral situation of the patient during treatment, whether they realize the temporary tooth in an implant-supported or non-implant-supported manner.

In a preferred embodiment, it is provided that a preferably metallic bushing is inserted into the drilled guide. The bushing is used for guiding the drill and prevents damage to the temporary tooth due to drill abrasion. The bushing can be removable. If it is expedient, the bushing may also protrude out of the temporary tooth.

In order to prevent the drill from penetrating to deeply, removable stop elements can be provided in an edge region of the drill guide for drilling depth limitation of the drill. These stop elements are advantageously connected by means of a detachable connection to the drill jig, particularly to the temporary tooth.

For aesthetic reasons, it is beneficial if at least one surface of the temporary tooth is made from opaque, preferably white or tooth coloured material. In particular, the production of the drill jig is simplified if the entire temporary tooth or even the entire drill jig consists of the same opaque material.

In order to be able to use a plurality of tools during the intervention simultaneously, it is advantageous if the temporary tooth comprises a lock for a tool such as a cup for example. In this context, cups are dental tools, which reduce the internal diameter of the drill guide, so that various drills, such as centre drills, pilot drills, depth drills, counterbores and screw taps with respectively different diameters can be guided precisely. According to the invention, these tools can be connected to the lock and locked as a result. The lock may be realized as a bayonet closure for this purpose.

The invention is explained in more detail in the following on the basis of preferred embodiments, to which it is not limited however.

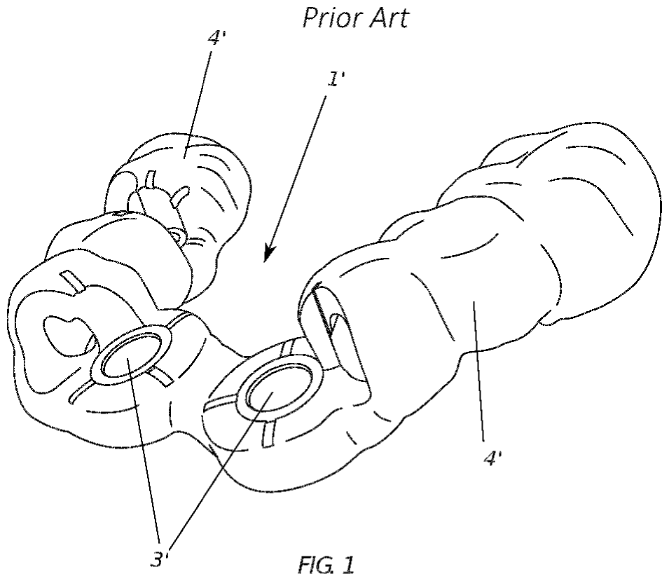

FIG. 1 shows a drill jig, as is known from the prior art.

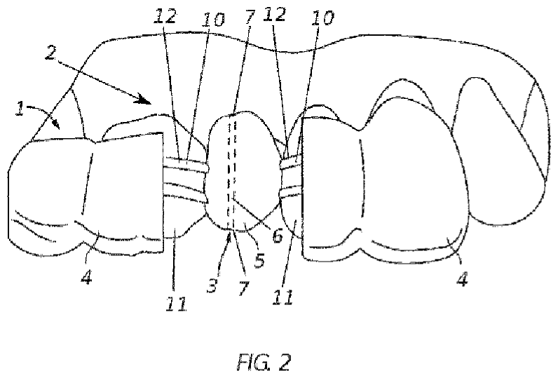

FIG. 2 shows a drill jig according to the invention on a dentition of a patient in a first embodiment.

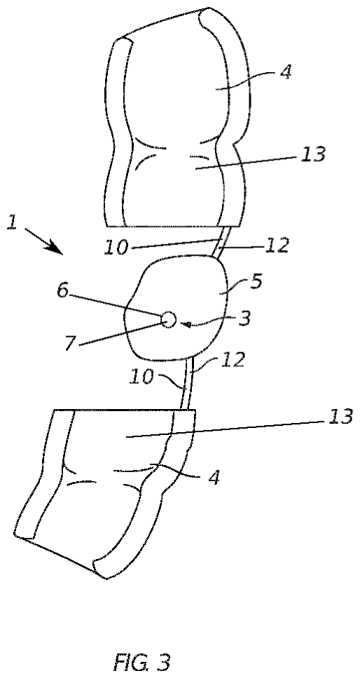

FIG. 3 shows the drill jig according to the invention of the first embodiment in a different view.

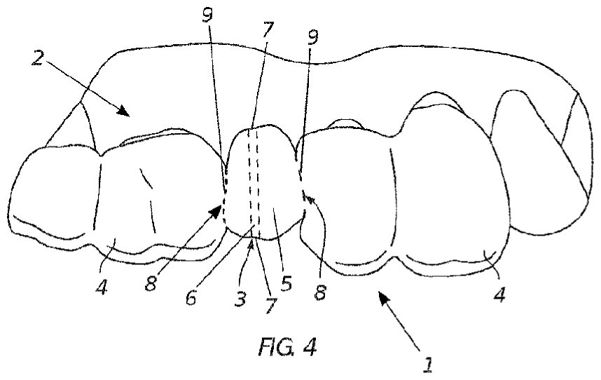

FIG. 4 shows a drill jig according to the invention on a dentition of a patient in a second embodiment.

For comparison, FIG. 1 shows a drill jig 1′ already known from the prior art, with a plurality of through holes as drill guides 3′. To fix the drill jig 1′ on a dentition of a patient (not shown), the drill jig has holding elements 4′, which are adapted to the teeth of the patient. During an oral surgery intervention, the drill jig 1′ is placed onto the dentition and a drill is guided through the drill guide 3′, in order to place the drill holes for the dental implants. After the intervention, the drill jig 1′ is removed and the fitted implants are protected using a separately manufactured temporary tooth (not shown). In contrast to the drill jig according to the invention (cf. FIGS. 2 to 4), drill jigs from the prior art do not have an integrated temporary tooth and can therefore be disposed of as a whole following the drilling as they cannot be put to any further use.

By comparison, FIG. 2 shows the drill jig 1 according to the invention in a first embodiment, which in the illustration shown is placed on a dentition 2 of a patient. For fixing, the drill jig 1 advantageously has two holding elements 4, between which a temporary tooth 5 is arranged. In FIG. 2, the temporary tooth 5 is inserted between two natural teeth of the dentition 2. In order to guide the drill onto the jaw of the patient during the intervention, the temporary tooth 5 comprises a drill guide 3. Preferably, the drill guide 3 is realized as a channel 6, which leads from a first side of the temporary tooth 5 to an opposite second side and penetrates the temporary tooth 5. Expediently, the channel 6 is straight, i.e. free of curvature, and closed except for two end-side openings 7 on the opposite sides of the temporary tooth 5. The dashed lines indicate that the drill guide 3 essentially runs inside the temporary tooth 5 and is only accessible through the end-side openings 7.

After drilling, the drill jig 1 can be taken from the dentition of the patient and the temporary tooth 5 can be detached from the holding elements 4 by hand by means of repeated bending or with the aid of a corresponding tool. For easier detachability, the temporary tooth 5 can be connected to the holding elements 4 by means of a detachable connection. In particular, it is beneficial to this end if the detachable connection is formed by a material constriction or a perforation (cf. FIG. 4). After the detachment of the temporary tooth 5 from the holding elements 4, the temporary tooth 5 can be placed at the position provided therefor in the dentition 2 of the patient. This is generally the position at which the temporary tooth 5 was placed during the intervention for guiding the drill. For aesthetic reasons, at least one side of the temporary tooth 5 can be produced from tooth-coloured material. To simplify the production, the entire drill jig 1 may be manufactured from the same tooth-coloured material.

To fasten the temporary tooth 5 on the dentition 2 of the patient after removal of the holding elements, the temporary tooth 5 can, as in the exemplary embodiment shown, comprise at least one holding web 10 for connection to a tooth surface 11, wherein the holding web 10 is preferably formed by a connection section 12 of the temporary tooth 5 to a holding element 4. The temporary tooth of the first embodiment is therefore particularly suitable as a non-implant-supported temporary tooth. The detachable connection between the temporary tooth 5 and a holding element 4 is preferably arranged on the end of the holding web 10 assigned to the corresponding holding element 4.

For improved fixing of the drill jig 1 during the intervention, it is beneficial if the holding elements 4 are formed for placement onto one or more teeth for positioning the drill jig 1 on the dentition 2 of the patient. This can be seen in particular from FIG. 3, which shows the drill jig 1 in a view from below, i.e. with a view onto the side facing the dentition 2 of the patient in the use state. In particular, the at least one holding element 4 can comprise an opening 13, preferably an opening 13 adapted to one or more teeth of the dentition 2 of the patient, for positioning the drill jig 1 on the dentition 2 of the patient. This opening 13 can be formed inter alia by means of an open cavity or a bulge of the holding element 4, which on the inside, i.e. the side facing the dentition in the use state, represents a negative or one or more teeth of the patient.

A second embodiment of the drill jig 1 is illustrated in FIG. 4. Like the first embodiment, the temporary tooth 5 is inserted between two holding elements 4 and comprises a drill guide 3 realized as a channel 6. Instead of the connection sections 12 or holding webs 10 described above in connection with FIG. 2, the temporary tooth 5 is connected directly to the holding elements 4 by means of a detachable connection 8, which connection may be formed inter alia by means of a material constriction or a perforation 9.

The temporary tooth 5 of the second embodiment is suitable for use as an implant-supported temporary tooth 5 in particular. For this purpose, the temporary tooth 5 may comprise a connection point (not shown) adapted to the implant on a side facing the jaw of the patient in the inserted state for the indirect or direct connection to an implant. Advantageously, the connection point may comprise a thread, using which the temporary tooth 5 can be connected to the implant. Preferably, the connection point is realized in such a manner in this case that in the connected state to the implant, the temporary tooth 5 is at least partially rotatable with respect to the implant, in order to enable subsequent adjustments of the temporary tooth 5 with regards to its position. An intermediate piece may also be arranged as an adapter between the connection point and the implant, wherein the connection point is adapted to the intermediate piece in this case.

1. A drill jig for an oral surgery on a patient, the drill jig comprising:
a temporary tooth, in which a drill guide is provided for guiding a drill into the jaw of the patient, and
at least one holding element connected to the temporary tooth for temporary fastening of the drill jig on the dentition of the patient wherein the at least one holding element is connected to the temporary tooth by a detachable connection, wherein the at least one holding element comprises an opening adapted to one or more teeth of the dentition of the patient for positioning the drill jig on the dentition of the patient.

2. The drill jig according to claim 1, wherein the detachable connection is formed by a material constriction or a perforation.

3. The drill jig according to claim 1, wherein the temporary tooth comprises at least one holding web for connection to a tooth surface.

4. The drill jig according to claim 3, wherein the holding web is preferably formed by a connection section of the temporary tooth to the at least one holding element.

5. The drill jig according to claim 1, wherein the temporary tooth comprises a connection point on a side facing the jaw of the patient in the inserted state for the indirect or direct connection to an implant.

6. The drill jig according to claim 1, wherein a bushing is inserted into the drill guide.

7. The drill jig according to claim 6, wherein the bushing is metallic.

8. The drill jig according to claim 1, wherein removable stop elements are provided in an edge region of the drill guide for drilling depth limitation of the drill.

9. The drill jig according to claim 1, wherein at least one surface of the temporary tooth is formed by an opaque material.

10. The drill jig according to claim 9, wherein the opaque material is a tooth coloured material.

11. The drill jig according to claim 1, wherein the temporary tooth comprises a lock for a tool.

12. The drill jig according to claim 11, wherein the tool is a cup.

13. A method for an oral surgery on a patient, comprising the steps of
obtaining a drill jig for the oral surgery, the drill jig comprising a temporary tooth in which a drill guide is provided for guiding a drill into the jaw of the patient and at least one holding element connected to the temporary tooth for temporary fastening of the drill jig on the dentition of the patient, wherein the at least one holding element comprises an opening adapted to one or more teeth of the dentition of the patient for positioning the drill jig on the dentition of the patient,
positioning the drill jig on the dentition of the patient by means of the opening adapted to the one or more teeth of the dentition of the patient,
drilling into the jaw of the patient through the drill guide provided in the temporary tooth,
inserting an implant into the jaw of the patient,
detaching the holding element from the temporary tooth, and
fastening the temporary tooth on the dentition of the patient or on the implant inserted into the jaw of the patient.

14. The method according to claim 13, further comprising the step of: upon wound healing, replacing the temporary tooth with a permanent dental prosthesis.

15. A method for an oral surgery on a patient, comprising the steps of
obtaining a drill jig for the oral surgery, the drill jig comprising a temporary tooth in which a drill guide is provided for guiding a drill into the jaw of the patient and at least one holding element connected to the temporary tooth for temporary fastening of the drill jig on the dentition of the patient,
positioning the drill jig on the dentition of the patient,
drilling into the jaw of the patient through the drill guide provided in the temporary tooth,
inserting an implant into the jaw of the patient,
detaching the holding element from the temporary tooth, and
fastening the temporary tooth on the dentition of the patient or on the implant inserted into the jaw of the patient.

16. The method according to claim 15, further comprising the step of: upon wound healing, replacing the temporary tooth with a permanent dental prosthesis.
